(19) United States
(12) Patent Application Publication
(10) Pub. No.: US 2018/0047774 A1
(43) Pub. Date: Feb. 15, 2018

(54) OPTOELECTRONIC DEVICE

(71) Applicant: ALCATEL LUCENT, Boulogne-Billancourt (FR)

(72) Inventors: Alexandre GARREAU, Palaiseau (FR); Romain BRENOT, Palaiseau (FR); Raphael AUBRY, Palaiseau (FR)

(21) Appl. No.: 15/552,945

(22) PCT Filed: Feb. 25, 2016
(86) PCT No.: PCT/EP2016/053993

§ 371 Date: Aug. 23, 2017

(30) Foreign Application Priority Data
Feb. 25, 2015 (EP) 15305288.1

(51) Int. Cl. H01L 27/146; H01L 31/0352; H01S 5/50; H01L 31/105; A61N 1/05; A61F 9/00; H01L 31/0304

(52) U.S. Cl. CPC H01L 27/14643; A61F 9/0017; H01L 31/035236; H01L 27/14694; H01L 31/03048; H01L 31/105; A61N 1/0543; H01S 5/50; G02B 5/3066

(57) ABSTRACT

The optoelectronic device includes a matrix of optoelectronic components including semiconductor optical amplifiers SOAs, the semiconductor optical amplifiers SOAs containing an active layer of gallium nitride GaN having multiple InGaN/GaAsN or InGaN/AlGaN quantum wells on a substrate of p-doped gallium nitride and covered with a layer of n-doped gallium nitride. The p-doped gallium nitride GaN substrate forms a column of p-GaN covered with a layer of an insulator in biocompatible material. The device can include a matrix having multiple electronic components of different heights. The optoelectronic component can be a photodiode or a semiconductor optical amplifier SOA. This optoelectronic device can be used in epiretinal or subretinal prostheses. A single epiretinal or subretinal prosthesis can include a matrix of photodiodes and a matrix of semiconductor optical amplifiers SOAs.

### FIELD

This invention concerns the field of implantable optoelectronic devices that can be used in particular in retinal prostheses designed to offset the deterioration of the photoreceptor cells of the human eye.

### BACKGROUND

The human eye, or eyeball, is a hollow structure with a globally spherical form. The innermost layer of the back part of the eye is the retina. The retina is a nervous structure, comprising many photoreceptors and neurones that process and channel visual information to the brain via the optic nerve. At the point where the optic nerve comes out, the retina is interrupted: this is the blind spot, close to which is the yellow spot, or macula, containing a central pit, known as the fovea.

Specialised photoreceptor nerve cells line the inner wall of the back of the eye; cones and rods, thus named due to their shape, which contain photo-sensitive is substances. The rods, sensitive to light intensity, are photoreceptors that are designed specifically for twilight vision and the cones, responsible for colour vision, are designed more specifically for daylight vision. Cones are divided into three families of cells, each with its sensitivity peak in a determined zone of the spectrum (blue-purple, green and yellow-green).

The deterioration of the photoreceptor cells of the human eye may be due for example to age-related macular degeneration (AMD) or to genetically inherited retinitis pigmentosa. The photoreceptors (cones and rods) are the cells of the retina that are sensitive to light, whereas the other neurones that process signals captured by photoreceptors send information to the brain via the optic nerve. When photoreceptor cells deteriorate, the retina can no longer respond to light. However, a sufficient number of other neurones remain so that their electrical stimulation produces the perception of light by the brain.

In order to treat the deficiencies of the photoreceptor cells, two methods have been explored: implanting retinal prostheses and the optogenetic approach.

The optogenetic technique involves changing the neurones to make them sensitive to light, by incorporating a light-sensitive protein into the cellular membrane. By making each cell sensitive to light, vision can potentially be restored to near-normal acuity. However, artificial vision based on the optogenetic approach presents a major drawback. The modified cells require blue (460 nm) and very bright light to be activated, and the light intensity required is around seven times greater than the light sensitivity threshold normally observed in healthy individuals.

Retinal prostheses have an optoelectronic device that includes a matrix of optoelectronic components that are activated, either by light entering the eye in the case of a “subretinal” prosthesis, or by an electric signal from a micro-camera fitted outside the eye, in the case of an “epiretinal” prosthesis. The different types of implants used require a silicon-based technology which is easy to use and allows the development of nanometric devices. However silicon is a material that is opaque in the visible optic field.

The epiretinal solution involves placing an electronic implant in the front of the retina to stimulate the neurones. The epiretinal implant itself is not sensitive to light and must be connected to a micro-camera fitted outside the eye. The epiretinal implant requires a “coder” whose function is to fulfil the role of the neurones in the inner layer of the retina, which perform the preliminary processing of visual information.

In the United States, in February 2013 the Food and Drug Administration (FDA) authorised the use of the first epiretinal prosthesis designed to treat patients with advanced retinitis pigmentosa. This epiretinal prosthesis known as the “Argus II Retinal Prosthesis System” is manufactured by the company Second Sight Medical Products Inc. This epiretinal prosthesis is a gold standard with a matrix of some sixty electrodes, and has already been tested on patients throughout the world.

The sub-retinal prosthesis is placed beneath the retina to replace the destroyed photoreceptor nerve cells, which is surgically more difficult to perform but allows the neurones to be stimulated in a more natural position. The subretinal prosthesis converts incidental light to an electric signal which is transmitted to the neurones (bipolar cells). The subretinal prosthesis is itself sensitive to light and does not need an external device. The reference subretinal prosthesis “Retina” is manufactured by the company Retina Implant AG.

It is considered that to read a text, to move about independently and to recognise a face, minimum resolution must be more than 1,000 pixels. The subretinal prosthesis is sensitive to light with a matrix of 1,500 pixels. With an epiretinal prosthesis, resolution is only 60 pixels. However, clinical tests performed with the two types of prosthesis give equivalent results, whereas there are a significantly larger number of electrodes in a subretinal prosthesis. When the number of optoelectronic components increases, the matrices become denser with smaller optoelectronic components. More precise positioning of the individual optoelectronic components becomes essential to increase the proximity between the optoelectronic component and the layer of retinal ganglion cells. This allows each optoelectronic component to activate a small portion of the retina to increase visual acuity.

Moreover, known retinal prostheses are flat two-dimensional (2D) devices that are not capable of making a three-dimensional (3D) simulation, which is a significant limitation of their performance.

### SUMMARY

More effective solutions than the current ones, without the afore-mentioned drawbacks, are therefore needed.

The solution proposed is a retinal prosthesis featuring a matrix of optoelectronic components with semiconductor optical amplifiers SOAs, which contain an active layer of gallium nitride GaN with multiple quantum wells InGaN/GaAsN (gallium indium nitride/gallium arsenide nitride) or InGaN/AlGaN (gallium indium nitride/gallium aluminium nitride) on a substrate of gallium nitride GaN with p-type doping and covered with a layer of gallium nitride GaN with n-type doping.

The semiconducting material GaN has the advantage of having good chemical stability and bio-compatibility. For this reason, it is possible to encapsulate materials that are not well tolerated by the human organism in this material since it creates a protective barrier.

The semiconducting material GaN also has the characteristic of being transparent in the wavelength range of visible light. In this way, the retina cells that are still functional are not affected by the opacity of the retinal prosthesis. Furthermore, the retina cells not affected are still stimulated since the retinal prosthesis does not mask the visible light penetrating the eye.

Thus the optoelectronic devices with a matrix containing optoelectronic components based on a gallium nitride GaN structure with Multi Quantum Wells (MQW) InGaN/GaAsN or InGaN/AlGaN present the advantage of letting the light pass between two neighbouring optoelectronic components.

From one viewpoint, the substrate of gallium nitride (GaN) with p-type doping forms a column of pGaN.

From another viewpoint, the column of p-GaN is covered with an insulating layer of bio-compatible material chosen from carbon, diamond, titanium dioxide TiO₂, silicon SiO₂, silicon nitride Si₃N₄ or gallium nitride GaN.

From yet another viewpoint, the ratio between the height and the cross dimension of the pGaN column is less than 20.

According to one method of construction, the optoelectronic device has a matrix of optoelectronic components that comprises semiconductor optical amplifiers SOAs of different heights.

According to another method of construction, the matrix of optoelectronic components has semiconductor optical amplifiers SOAs with vertical cavity or semiconductor optical amplifiers SOAs with horizontal cavity. When the matrix of optoelectronic components has at least one semiconductor optical amplifier SOA with vertical cavity, two distributed Bragg reflectors are placed on each side of the active GaN layer with multi quantum wells so that an optical cavity is defined.

According to yet another method of construction, the matrix of optoelectronic components is a three-dimensional (3D) matrix of semiconductor optical amplifiers SOAs with vertical cavity or semiconductor optical amplifiers SOAs with horizontal cavity.

The semiconductor optical amplifiers SOAs should be spaced at distance E such that E²=π(350/2)²×1/n where n is the number of optoelectronic components in the matrix.

A transparent matrix of semiconductor optical amplifiers SOAs amplifies the blue, yellow or green light to improve the results obtained with the optogenetic technique, or with epiretinal or subretinal prostheses.

According to one method of construction, the optoelectronic component is a photodiode. The use of a transparent matrix of photodiodes with multi quantum wells

InGaN/GaAsN or InGaN/AlGaN eliminates the need for the micro-camera used today with the epiretinal prosthesis.

According to another method of construction, the matrix of optoelectronic components also has vertical or horizontal photodiodes. The matrix of optoelectronic components should preferably contain at last one photodiode and at least one semiconductor optical amplifier SOA.

According to another method of construction, the optoelectronic device has a matrix of optoelectronic components that comprises vertical or horizontal photodiodes of different heights. The photodiodes and semiconductor optical amplifiers SOAs are of different heights in order to more accurately stimulate the layer of retinal ganglion cells and/or the optical nerve.

The vertical or horizontal photodiodes should be spaced at distance E such that E²=π(350/2)²×1/n where n is the number of optoelectronic components in the matrix.

A retinal prosthesis, which is an epiretinal prosthesis, is also proposed.

A retinal prosthesis, which is a subretinal prosthesis, is also proposed.

A retinal prosthesis featuring both a subretinal prosthesis and an epiretinal prosthesis, is also proposed.

According to one viewpoint, an epiretinal or subretinal prosthesis features a matrix with at least one vertical photodiode.

According to a second viewpoint, an epiretinal or subretinal prosthesis features a matrix with at least one horizontal photodiode.

According to a third viewpoint, an epiretinal or subretinal prosthesis features a matrix with at least one semiconductor optical amplifier with vertical cavity.

According to a fourth viewpoint, an epiretinal or subretinal prosthesis features a matrix with at least one semiconductor optical amplifier with horizontal cavity.

According to yet another viewpoint, at the same time at least one matrix of photodiodes and at least one matrix of semiconductor optical amplifiers SOAs can be incorporated into the same epiretinal or subretinal prosthesis in order to stimulate the neurones by both injecting an electric signal and amplifying the blue, green or yellow light.

### BRIEF DESCRIPTION

Other characteristics and advantages of the present invention will become apparent upon reading the following description of embodiments, naturally given by way of illustrative and non-limiting examples, and in the attached drawing in which

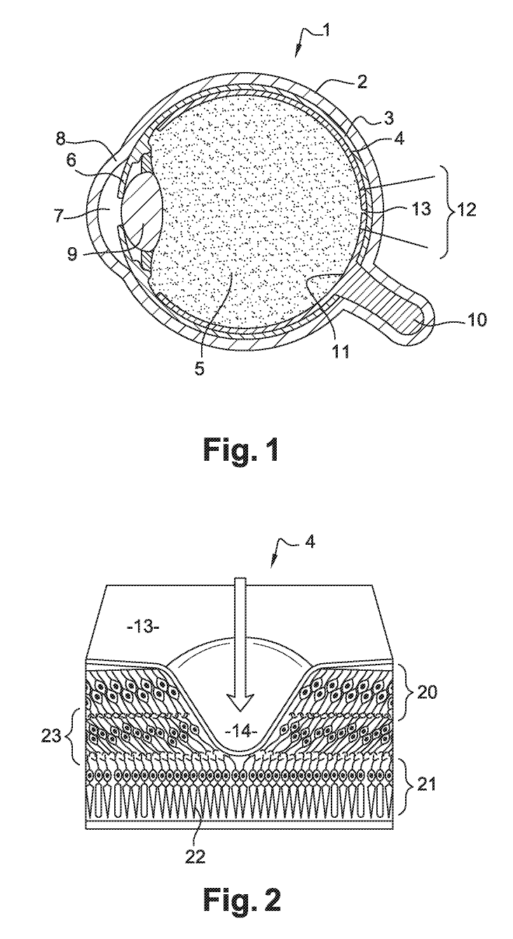

FIG. 1 illustrates a schematic cross-sectional view of a human eye

FIG. 2 illustrates a schematic cross-sectional view of the retina

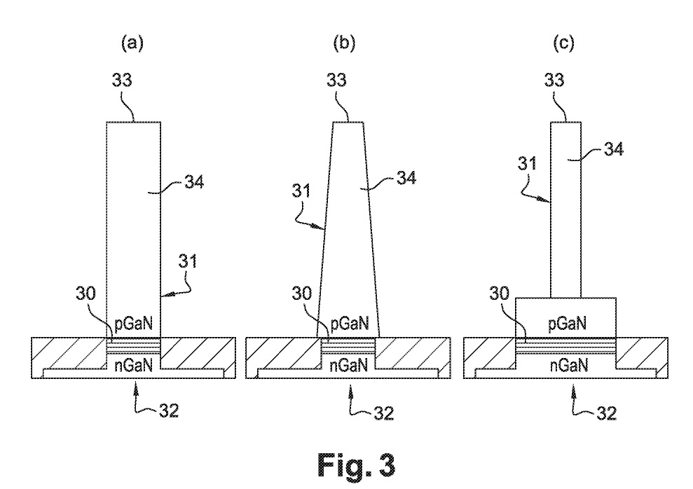

FIGS. 3a, 3b and 3c illustrate schematically an embodiment of an optoelectronic is component according to the invention

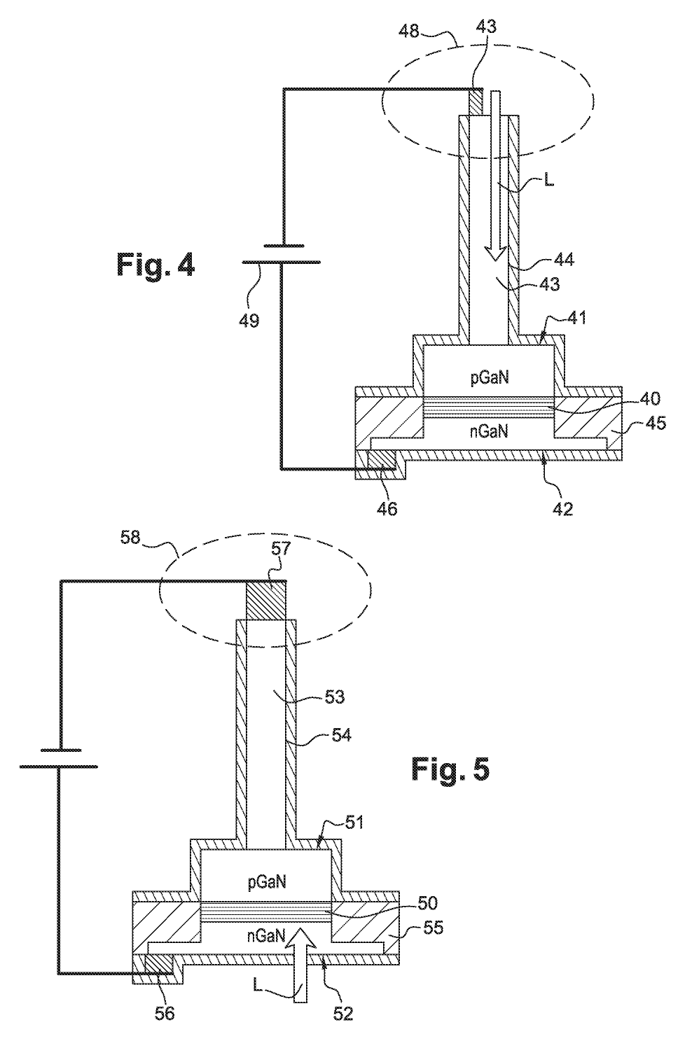

FIG. 4 illustrates schematically an embodiment of an optoelectronic device with a vertical photodiode applicable to a subretinal prosthesis

FIG. 5 illustrates schematically an embodiment of an optoelectronic device with a vertical photodiode applicable to an epiretinal prosthesis

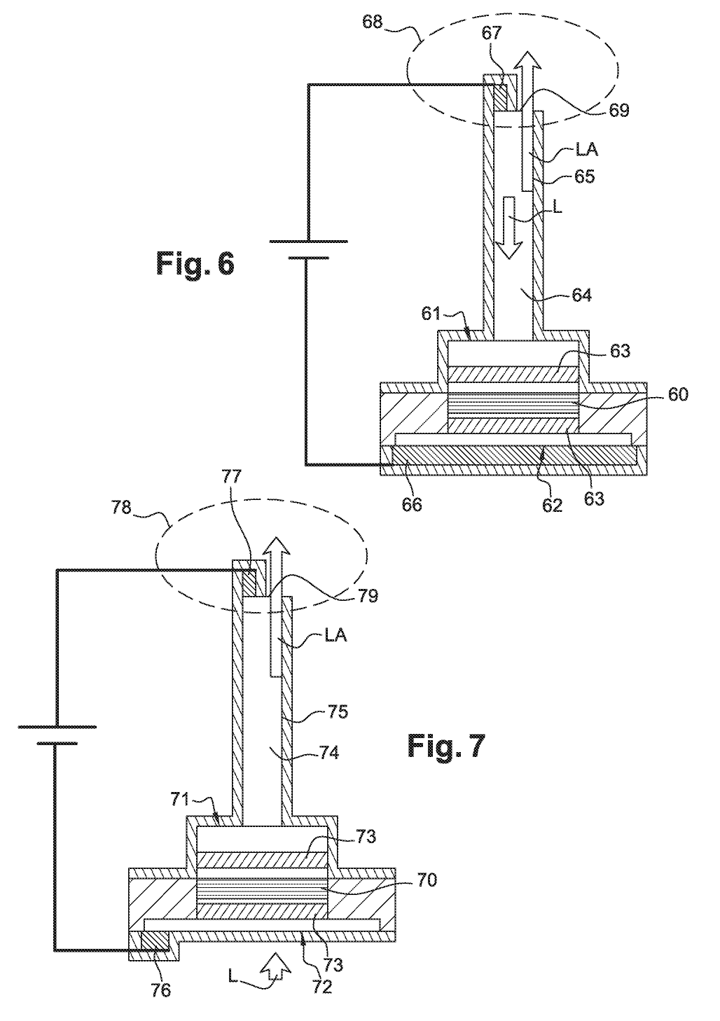

FIG. 6 illustrates schematically an embodiment of an optoelectronic device with a semiconductor optical amplifier with vertical cavity applicable to a subretinal prosthesis

FIG. 7 illustrates schematically an embodiment of an optoelectronic device with a semiconductor optical amplifier with vertical cavity applicable to an epiretinal prosthesis

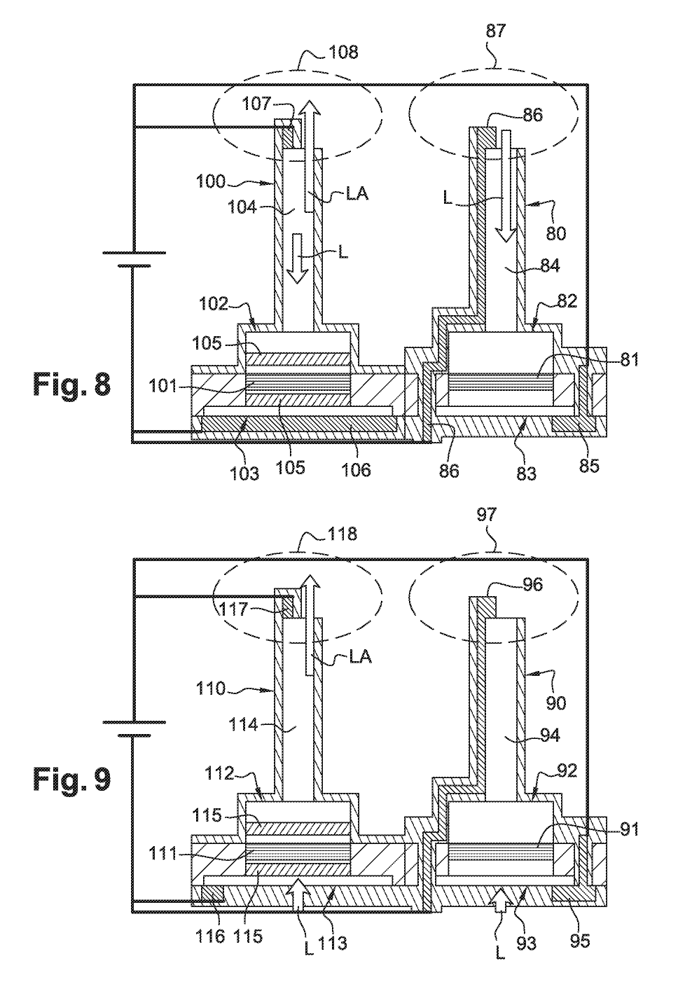

FIG. 8 illustrates schematically an embodiment of an optoelectronic device with a vertical photodiode and a semiconductor optical amplifier with vertical cavity applicable to a subretinal prosthesis

FIG. 9 illustrates schematically an embodiment of an optoelectronic device with a vertical photodiode and a semiconductor optical amplifier with vertical cavity applicable to an epiretinal prosthesis

FIG. 10 illustrates schematically an embodiment of a matrix of optoelectronic components

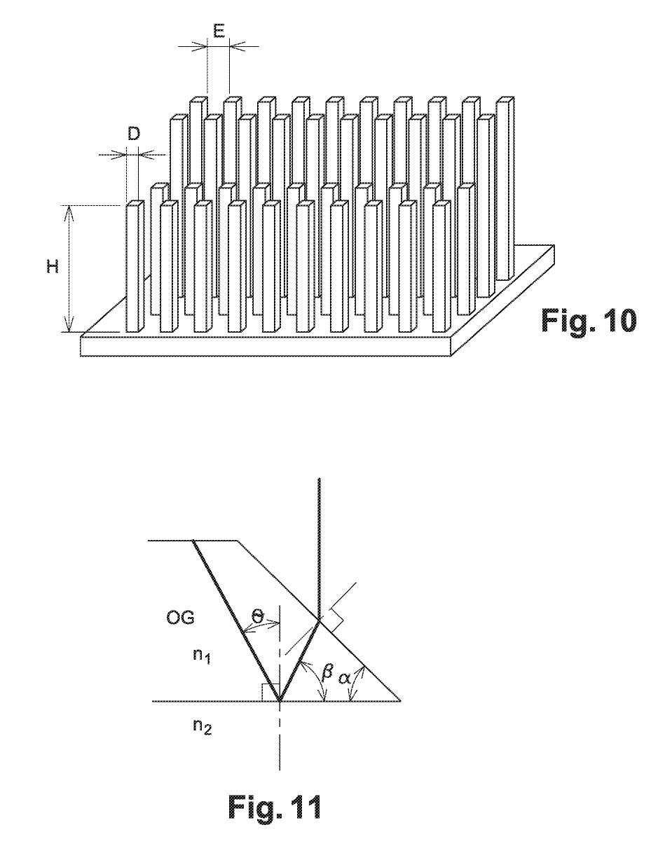

FIG. 11 illustrates the facet of the horizontal cavity GaN, for photodiode GaN and semiconductor optical amplifier SOA applications

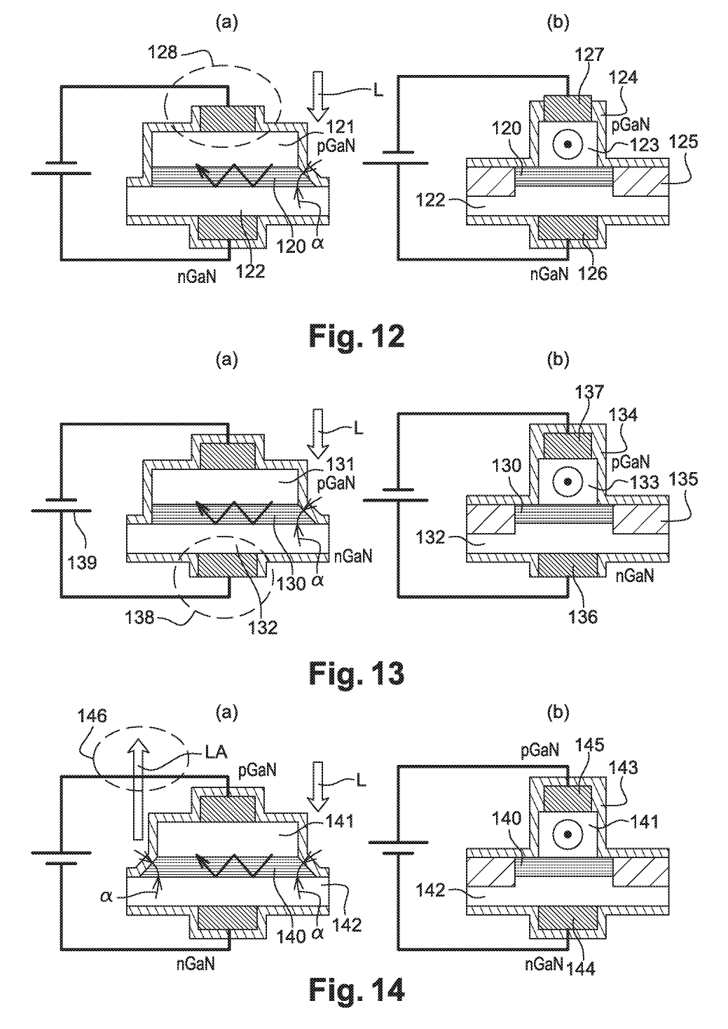

FIGS. 12a and 12b illustrate schematically two perpendicular side views of one embodiment of an optoelectronic device consisting of a horizontal photodiode that can be applied to a sub-retinal prosthesis,

FIGS. 13a and 13b illustrate schematically two perpendicular side views of one embodiment of an optoelectronic device consisting of a horizontal photodiode that can be applied to an epiretinal prosthesis,

FIGS. 14a and 14b illustrate schematically two perpendicular side views of one embodiment of an optoelectronic device consisting of a semiconductor optical amplifier with horizontal cavity that can be applied to a sub-retinal prosthesis,

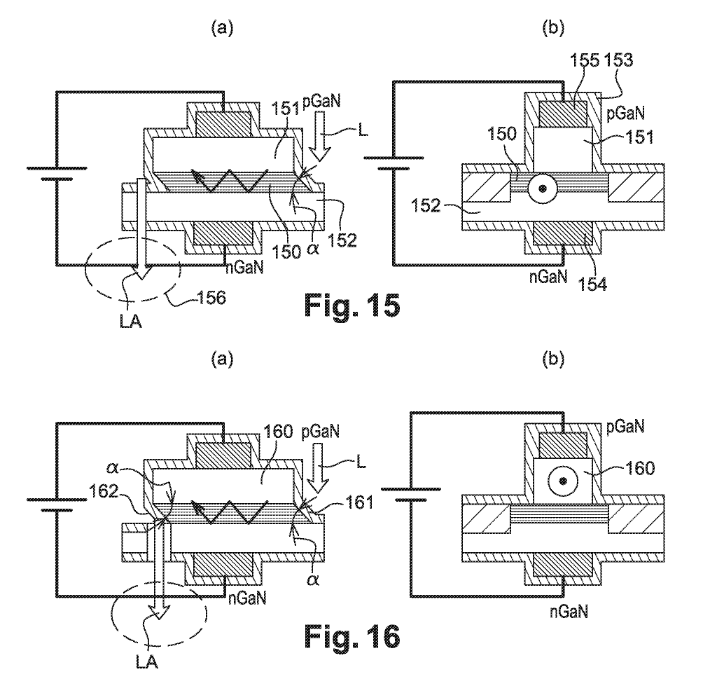

FIGS. 15a and 15b illustrate schematically two perpendicular side views of one embodiment of an optoelectronic device consisting of a semiconductor optical amplifier with horizontal cavity that can be applied to an epiretinal prosthesis,

FIGS. 16a and 16b illustrate schematically two perpendicular side views of another embodiment of an optoelectronic device consisting of a semiconductor optical amplifier with horizontal cavity that can be applied to an epiretinal prosthesis.

Directional terminology like “left” and “right”, “top” and “bottom”, “front” and “rear”, “horizontal” and “vertical”, “above” and “below”, etc., is used here with reference to the orientation of the figures described. Since the components that make up the embodiments may be placed in different orientations, the directional terminology is used here only for illustrative purposes and is in no way limiting.

### DETAILED DESCRIPTION

FIG. 1 illustrates schematically a cross-section of a human eye 1. It is composed of three superposed membranes 2, 3, 4 surrounding a gelatinous substance called the vitreous humour 5.

The anterior chamber of the eye, which receives the light, consisting of
the iris 6 with a round opening in its centre called the pupil 7, which allows light to pass into the eye and the size of which adapts automatically to the brightness the eye is exposed to,
the cornea 8, a round, transparent, domed membrane that allows light rays to pass through,
the lens 9, which focuses the image on the retina depending on the distance.

The retina 4 is the membrane that lines the inner surface of the eye's posterior chamber. The retina's nerve cells convert the light energy into electrical signals, which are transmitted to the brain by the optic nerve 10. The blind spot 11 is the area of the eye where the fibres meet to form the optic nerve, and which contains no photosensitive cells. Nearby, the macula 12 (or yellow spot) is formed of numerous visual cells.

The most sensitive area of the retina, devoid of any blood capillaries, is called the fovea 13. The fovea 13 is a small part of the retina found in the macula 12 (approximately 6 mm in diameter) that is sensitive to colours and is important for visual acuity. The foveola 14 (approximately 0.35 mm in diameter) is located in the middle of the fovea 13 (approximately 1.5 mm in diameter) and contains only cone cells. The fovea 13 is the part of the retina 4 with the highest visual acuity—this is where the rays of light have entered directly with the least interference, and is where the density of photoreceptor cells is at its highest. In the foveola 14, the photoreceptor cones are longer, thinner, and more densely packed than elsewhere in the retina 4. This ensures is the foveola 14 has the highest visual acuity in the retina 4. The photoreceptor cells convert the light energy into nervous impulses that are sent to the optic nerve.

As illustrated in the schematic cross-section view in FIG. 2, the retina 4 is composed of a stack of different layers arranged radially at the fovea 13. The outer layer 20, the layer of retinal ganglion cells (RGCs), stops the light from diffusing inside the eye. The inner layer 21, the layer of photoreceptors (PRs), is formed of specialised nerve receptor cells 22, with the rods and cones detecting light and the neurons processing and transmitting the visual information to the brain. The inner layer 22 is directly accessible by the foveola 14. The middle layer 23, or inner nuclear layer (INL), contains connecting cells such as bipolar cells.

There are several kinds of retinal prosthesis that use an optoelectronic device consisting of optoelectronic components based on a common concept as illustrated by FIGS. 3a to 3c. This concept is based on carrying out one or more epitaxies on an intrinsic active GaN layer 30 with multiple quantum wells for InGaN/GaAsN (indium gallium nitride/arsenic gallium nitride) or InGaN/AlGaN (indium gallium nitride/aluminium gallium nitride) on a substrate 31 of p-type doped gallium nitride GaN. An intrinsic material is a semiconductive material that is not doped and/or has no impurities. Epitaxy is the crystalline growth of a material, generally carried out on the same material respecting the crystals' meshing and orientation. At the top of each active GaN layer 30, a layer of n-type doped GaN gallium de layer 32 is carried out to complete the epitaxy.

The process applied to the rear surface 33 of the p-GaN substrate 31, wherein the p-GaN substrate 31 is thinned and polished to the desired height, results in p-GaN columns 34. The p-GaN columns 33 are obtained by selective etching of the p-GaN substrate layer 31, for example with a chloride inductively coupled plasma ICP.

The p-GaN column 34 must be long enough to stimulate the cells of the retina. The ratio between the height and transverse measurement (width or diameter) of the p-GaN column 34 should preferably be less than 20, to prevent the column from breaking. The p-GaN columns 34 may be different heights in order to stimulate different layers of the retina. The different heights are achieved through selective etching of the p-GaN substrate 31 for example, by starting from the rear surface. The p-GaN column 34 may take the shape of a rod with parallel edges (FIG. 3a), a truncated pyramid (FIG. 3b), or a thin rod on top of a wider base (FIG. 3c). In the remainder of this description, we shall consider a thin rod on top of a wider base, as shown in FIG. 3c.

Several optoelectronic components with a similar structure consisting of multiple quantum wells can be created using selective area growth (SAG) technology, in order to amplify or detect several wavelengths (blue, green, yellow). The various optoelectronic components found on the same matrix are electrically separated by an area of implanted GaN or semi-isolating GaN, so that the optoelectronic components are isolated from each other and the matrix remains transparent.

FIG. 4 illustrates a schematic representation of an optoelectronic device embodiment, composed of at least one GaN-based vertical photodiode and intended for use in a sub-retinal prosthesis.

An absorbent active GaN layer 40 with multiple InGaN/GaAsN or nGaN/AlGaN quantum wells is made by epitaxy on a substrate 41 of p-type doped gallium nitride GaN. A layer 42 of n-type doped gallium nitride GaN is laid on top of the absorbent GaN layer 40. By thinning and polishing the p-GaN substrate 41, a p-GaN column 43 is obtained at the desired height. The p-GaN column 43 is coated with an isolating layer 44 of dielectric or semi-isolating material. Furthermore, the material composing the isolating layer 44 must offer a good level of biocompatibility, such as carbon, diamond, titanium dioxide, common dielectric materials (silica, silicon nitride, etc.) or semi-isolating GaN material. The isolation is completed by implanting semi-isolating GaN 45 to separate the optoelectronic components from each other, in order to polarise the optoelectronic components in a matrix independently.

A metal contact 46 is placed on the front surface of the n-type doped gallium nitride GaN layer 42. The metal contact 46 on the front surface of the n-GaN layer 42 polarises the retinal prosthesis. Another metal contact 47 is placed at the end of the p-GaN column 43 corresponding with the area of the retina 48 that is stimulated. The metal contacts 46 and 47 are connected by an electrochemical generator 49 (battery or accumulator), which establishes a voltage between them. Because the light L must pass through the p-GaN column 43 to reach the absorbent active GaN layer 40, the metal contact 47 must not cover the entire upper surface of the p-GaN column 43. A photocurrent appears, which will stimulate the retina's various layers.

The embodiment schematically illustrated in FIG. 5 shows an optoelectronic device consisting of at least one GaN-based vertical photodiode that is designed for use with an epiretinal prosthesis.

An absorbent active GaN layer 50 with multiple InGaN/GaAsN or InGaN/AlGaN quantum wells is made by epitaxy on a substrate 51 of p-type doped gallium nitride GaN. A layer 52 of n-type doped gallium nitride GaN is laid on top of the absorbent GaN layer 50. By thinning and polishing the p-GaN substrate 51, a p-GaN column 53 is obtained at the desired height. The p-GaN column 53 is coated with an isolating layer 54 of dielectric or semi-isolating material. Isolation is completed by implanting semi-isolating GaN 55. Each photodiode in a matrix may be independently polarised from its neighbour in this way, depending on the medical requirement. A metal contact 56 is placed on the front surface of the n-GaN layer 52, and another metal contact 57 is placed at the end of the p-GaN column 53 that corresponds to the area of the retina 58 to be stimulated. Because the light L must pass through the nGaN column 52 to reach the absorbent active GaN layer 50, the metal contact 56 must not cover the entire front surface of the n-GaN column 52. A photocurrent appears, which will stimulate the retina's various layers. However, the metal contact 57 may cover the entire surface at the end of the pGaN column 53 because the induced photocurrent is enough to stimulate the different layers of the retina. Indeed, there is no need to transmit the light outside of the epiretinal area where there are no photoreceptor cells.

Replacing the retina with matrices containing thousands, if not millions, of optoelectronic components based on semiconductors, like these photodiodes, will make it possible to convert the light into an electrical signal, which will then be transmitted to the visual fibres that are still functioning.

We will now consider FIG. 6, which illustrates a schematic view of an optoelectronic device embodiment, composed of at least one GaN-based semiconductor optical amplifier with vertical cavity and intended for use in a sub-retinal prosthesis.

Remember that an optical amplifier is a device that amplifies an optical signal directly, without the need to convert it into an electrical signal beforehand. An optical amplifier is different from a laser in that it has no optical cavity, or there is no retroaction produced from the cavity. The semiconductor optical amplifiers SOAs are optical amplifiers that use semiconductive material to provide the gain medium. These semiconductor optical amplifiers SOAs contain anti-reflective parts at its end surfaces, which results in energy loss from the cavity that is above the gain, thus preventing the optical amplifier from working like a laser.

An amplifying active GaN layer 60 with multiple InGaN/GaAsN or InGaN/AlGaN quantum wells is made by epitaxy on a substrate 61 of p-type doped gallium nitride GaN. A layer 62 of n-type doped gallium nitride GaN is laid on top of the amplifying GaN layer 60. Two distributed Bragg reflectors DBRs 63 are placed on either side of the amplifying GaN layer 60.

By thinning and polishing the p-GaN substrate 61, a p-GaN column 64 is obtained at the desired height. The p-GaN column 64 is coated with an isolating layer 65 of dielectric or semi-isolating material.

A metal contact 66 is placed on the front surface of the n-GaN layer 62, and another metal contact 67 is placed at the end of the p-GaN column 64 that corresponds to the area of the retina 68 to be stimulated. Since on the one hand the incident light L must be able to penetrate the p-GaN substrate 61 to reach the amplifying GaN layer 60, and on the other hand the amplified light AL must be able to reach the area that requires stimulation 68, the metal contact 67 must not cover the entire surface at the end of the p-GaN column 64.

The distributed Bragg reflectors DBRs 63 define an optical cavity in which blue light is amplified. All of the blue light is reflected on the mirror created by the metal contact 66 covering the front surface of the n-GaN layer 62.

After carrying out an optogenetic operation, the retinal cells will be selectively stimulated by the amplified blue light AL. An anti-reflective coating 69 is necessary on the top end of the pGaN column 64 in order to prevent parasite reflections and improve the quality of optical transmission.

FIG. 7 illustrates a schematic view of an optoelectronic device embodiment, composed of at least one GaN-based semiconductor optical amplifier with vertical cavity and intended for use in an epiretinal prosthesis.

An amplifying active GaN layer 70 with multiple InGaN/GaAsN or InGaN/AlGaN quantum wells is made by epitaxy on a substrate 71 of p-type doped gallium nitride GaN. A layer 72 of n-type doped gallium nitride GaN is laid on top of the amplifying GaN layer 70. Two distributed Bragg reflectors DBRs 73 are placed on either side of the amplifying GaN layer 70.

By thinning and polishing the p-GaN substrate 71, a column of p-GaN of the desired height 74 is produced. The p-GaN layer 74 is covered with an insulating layer 75 of dielectric or semi-insulating material.

A metal contact 76 is placed on the front face of the n-GaN layer 72 and another metal contact 77 is placed at the end of the p-GaN column 74 corresponding to the area of retina 78 to be stimulated. Because the incident blue light L has to cross the n-GaN layer 72 to reach the GaN amplifying layer 70, the metal contact 76 must not cover the entire surface of the front face of the n-GaN layer 72. Once the blue light LA has been amplified it has to leave the column of p-GaN 74 to stimulate the neighbouring layers 78 of the retina, where the optogenetic therapy has been active, and the metal contact 77 must therefore not cover the entire surface of the end of the column of p-GaN 74.

The blue light is amplified in the optical cavity defined by the two distributed Bragg reflectors DBRs 73, positioned either side of the GaN amplification layer 70. After an optogenetic operation, the retina cells will be selectively stimulated by this amplified blue light LA. An anti-reflection coating 79 is required at the upper end of the p-GaN column 74 to prevent parasitic reflections, and to improve the optical transmission quality.

It may be advantageous to combine an optoelectronic device intended as a subretinal prosthesis with an optoelectronic device intended as an epiretinal prosthesis, whether or not they have the same operational mode. For example, a subretinal prosthesis containing optical amplifiers can be combined with an epiretinal prosthesis containing photodiodes, in particular in cases where optogenetic therapy proves more effective for cells close to the layer of ganglion cells than for photoreceptive cells such as cones. Or inversely, an epiretinal prosthesis containing optical amplifiers can be combined with a subretinal prosthesis containing photodiodes. It is also possible to combine photodiodes and optical amplifiers in a single epiretinal or subretinal prosthesis. This can be achieved by the use of vias (metallised holes) to produce direct and indirect polarisation of the optoelectronic components.

In the embodiment illustrated schematically in FIG. 8, an optoelectronic device containing at least one GaN-based photodiode and at least one vertical cavity GaN-based semiconductor optical amplifier combined, is intended for use in a subretinal prosthesis.

Photodiode 80, analogous to that in FIG. 4, contains an active absorbent GaN layer 81 with multiple InGaN/GaAsN or InGaN/AlGaN quantum wells deposited on a p-GaN substrate 82 and surmounted by an n-GaN layer 83 which is cut to form a column of p-GaN 84. A metal contact 85 is deposited on the n-GaN layer 83 and another metal contact 86 partially covers the upper end of the p-GaN column 84 corresponding to the area of retina 87 to be stimulated.

The vertical cavity semiconductor optical amplifier 100 contains a GaN amplifying layer 101 with multiple InGaN/GaAsN or InGaN/AlGaN quantum wells, deposited on a p-GaN substrate 102 and surmounted by a n-GaN layer 103 which is cut to form a column of p-GaN 104. Two distributed Bragg reflectors DBRs 105 are placed either side of the GaN amplifying layer 101. A metal contact 106 is deposited on the n-GaN layer 103 and another metal contact 107 partially covers the upper end of the p-GaN column 102 corresponding to the area of retina 108 to be stimulated.

The multiple quantum well structure of the photodiode and the multiple quantum well structure of the vertical cavity semiconductor amplifier can be adapted with distributed Bragg reflectors, by using butt-joint epitaxy.

We now consider FIG. 9, schematically illustrating an embodiment for an optoelectronic device containing at least one GaN-based photodiode and at least one vertical cavity GaN-based semiconductor optical amplifier combined, intended for use in an epiretinal prosthesis.

Photodiode 90, analogous to that in FIG. 5, contains an active absorbent GaN layer 91 with multiple InGaN/GaAsN or InGaN/AlGaN quantum wells deposited on a p-GaN substrate 92 and surmounted by an n-GaN layer 93 which is cut to form a column of p-GaN 94. A metal contact 95 is deposited on the n-GaN layer 93 and another metal contact 96 partially covers the upper end of the p-GaN column 94 corresponding to the area of retina 97 to be stimulated.

The vertical cavity semiconductor optical amplifier 110 contains a GaN amplifying layer 111 with multiple InGaN/GaAsN or InGaN/AlGaN quantum wells, deposited on a p-GaN substrate 112 and surmounted by an n-GaN layer 113 which is cut to form a column of p-GaN 114. Two distributed Bragg reflectors DBRs 115 are placed either side of the GaN amplifying layer 111. A metal contact 116 is deposited on the n-GaN layer 113 and another metal contact 117 partially covers the upper end of the p-GaN column 112 corresponding to the area of retina 118 to be stimulated.

It thus becomes possible to replace the retina by a prosthesis consisting of an optoelectronic device containing thousands or even millions of optoelectronic components in a matrix, as illustrated in FIG. 10.

An important parameter is the distance between two optoelectronic components in a matrix. There must be enough free space between the optoelectronic components for the active cells in the internal nuclear layer INL or the ganglion cell layer GCL to function normally. It may in particular be interesting to enable organic tissues to be introduced between the individual optoelectronic components, But there must also be a sufficient number of optoelectronic components (photodiodes or optical amplifiers) to allow the patient good image definition.

The foveola has a diameter of approximately 0.35 mm. The spacing E between two adjacent devices is given by the following relation, where n is the number of optoelectronic components in the matrix:

E²(μm)=ρ(350/2)²×1/n

In the case of a matrix with 2000 optoelectronic components, the spacing D is about 48 μm. The height H of the p-GaN column must be less than 480 μm, given that the thickness of the retina is generally less than 0.5 mm. In an optoelectronic device containing optoelectronic components in which the p-GaN column has a transverse dimension D (width or diameter) of about 24 μm, there remains 24 μm available to allow, for example, for metal contacts and electrical connections.

FIG. 11 schematically illustrates the facet of the horizontal GaN cavity, for GaN photodiode and semiconductor optical amplifier SOA applications. The facet is beveled at an angle a. To obtain total reflection on the guide layers of the MQW-based optical guide OG with an overall optical index n1 and the confinement layers with an overall optical index n2, the angle θ must be greater than the Brewster angle θ_{Brewster} and defined by the following inequalities:

n1>n2

θ>θ_{Brewster}

α>θ_{Brewster}

β<π/2−θ_{Brewster}

θ_{Brewster}=arc sin (n2/n1)

FIGS. 12a and 12b schematically illustrate an embodiment of an optoelectronic device, containing at least one GaN-based horizontal photodiode, intended for use in a subretinal prosthesis. FIG. 12a is a side view of the device in which light is propagated in the plane of the figure, and FIG. 12b is another side view of the device perpendicular to FIG. 12a.

An active absorbent GaN amplifying layer 120 with multiple InGaN/GaAsN or InGaN/AlGaN quantum wells is made by epitaxy on a p-doped gallium nitride GaN substrate 121. A layer 122 of n-doped gallium nitride GaN is deposited above the GaN absorbent layer 120. By selective etching of the p-GaN substrate 121 using an inductively coupled plasma ICP, a column of p-GaN 123 is formed up to the desired height, sufficient to allow stimulation of the retinal cells. The p-GaN layer 123 is covered with an insulating layer 124 of dielectric or semi-insulating material, Furthermore, the material composing the insulating layer 124 must have good biocompatibility, such as carbon, diamond, titanium dioxide, common dielectric materials (silica, silicon nitride, etc.) or the semi-insulating material GaN. The insulation is completed by implanting semi-insulating GaN 125 to separate the optoelectronic components from each other, to allow each of the optoelectronic components in the matrix to be polarised independently.

On the front face of the n-doped gallium nitride GaN layer 122, a metal layer 126 is deposited. The metal contact 126 on the front face of the n-GaN layer 122 allows the retinal prosthesis to be polarised. Another metal contact 127 is placed at the upper end of the p-GaN column 123 corresponding to the area of the retina 128 that is stimulated. The metal contacts 126 and 127 are connected by an electrochemical generator 129 (primary or rechargeable battery) which applies a voltage between them. Because the light L has to cross the p-GaN column 123 to reach the absorbent GaN amplifying layer 120, the metal contact 127 must not cover the entire surface of the front face of the p-GaN column 123. There appears a photocurrent which will stimulate the various layers of the retina.

In the embodiment illustrated in FIGS. 13a and 13b, an optoelectronic device containing at least one GaN-based horizontal photodiode intended for use in an epiretinal prosthesis is illustrated. FIG. 13a is a side view of the device in which light is propagated in the plane of the figure, and FIG. 13b is another side view of the device perpendicular to FIG. 13a.

An active absorbent GaN amplifying layer 130 with multiple InGaN/GaAsN or InGaN/AlGaN quantum wells is made by epitaxy on a p-doped gallium nitride GaN substrate 131. A layer 132 of n-doped gallium nitride GaN is deposited above the GaN absorbent layer 130. From the p-GaN substrate 131, a column of p-GaN of the desired height 133 is produced. The p-GaN layer 133 is covered with an insulating layer 134 of dielectric or semi-insulating material. The insulation is completed by implanting semi-insulating GaN 135. Each photodiode in a matrix can thus be polarised independently from its neighbour according to medical requirements. A metal contact 136 is placed on the front face of the n-GaN layer 132 and another metal contact 137 is placed at the end of the p-GaN column 133 corresponding to the area of retina 138 to be stimulated.

The light must enter through the beveled edge of the optical guide OG. The beveled edge inclined at an angle a has a TiO₂ and SiO₂-based anti-reflection coating that has been deposited to ensure good optical transmission between the exterior of the device and the guide layers. A photoelectric current appears, stimulating the various layers of the retina.

FIGS. 14a and 14b schematically illustrate an embodiment of an optoelectronic device, containing at least one horizontal cavity GaN-based semiconductor optical amplifier, intended for use in a subretinal prosthesis. FIG. 14a is a side view of the device in which light is propagated in the plane of the figure, and FIG. 14b is another side view of the device perpendicular to FIG. 14a.

An active GaN amplifying layer 140 with multiple InGaN/GaAsN or InGaN/AlGaN quantum wells is made by epitaxy on a p-doped gallium nitride GaN substrate 141. A layer 142 of n-doped gallium nitride GaN is deposited above the GaN amplifying layer 140. The p-GaN layer 141 is covered with an insulating layer 143 of dielectric or semi-insulating material.

A metal contact 144 is placed on the front face of the n-GaN layer 142 and another metal contact 145 is placed at the end of the p-GaN layer 141 corresponding to the area of retina 146 to be stimulated. Because part of the incident light L has to be able to cross the p-GaN substrate 141 to reach the GaN amplifying layer 140, and another part of the amplified light LA has to be able to reach the area to be stimulated 146, the metal contact 145 must not cover the entire surface of the end of the p-GaN layer 141. After an optogenetic operation, the retina cells will be selectively stimulated by the amplified blue light LA.

FIGS. 15a and 15b schematically illustrate an embodiment of an optoelectronic device, containing at least one horizontal cavity GaN-based semiconductor optical amplifier, intended for use in an epiretinal prosthesis. FIG. 15a is a side view of the device in which light is propagated in the plane of the figure, and FIG. 15b is another side view of the device perpendicular to FIG. 15a,

An active GaN amplifying layer 150 with multiple InGaN/GaAsN or InGaN/AlGaN quantum wells is made by epitaxy on a p-doped gallium nitride GaN substrate 151. A layer 152 of n-doped gallium nitride GaN is deposited above the GaN amplifying layer 150. The p-GaN layer 151 is covered with an insulating layer 153 of dielectric or semi-insulating material.

A metal contact 154 is placed on the front face of the n-GaN layer 152 and another metal contact 155 is placed at the end of the p-GaN layer 151 corresponding to the area of retina 156 to be stimulated. The incident blue light L must reach the GaN amplifying layer 150, and once the blue light LA is amplified it will stimulate the layers close to the retina. After an optogenetic operation, the retina cells will be selectively stimulated by this amplified blue light LA.

FIGS. 16a and 16b schematically illustrate another embodiment of an optoelectronic device, containing at least one semiconductor optical amplifier based on horizontal cavity GaN, intended for use in an epiretinal prosthesis. FIG. 16a is a side view of the device in which light is propagated in the plane of the figure, and FIG. 16b is another side view of the device perpendicular to the plane of FIG. 16a.

In this other version, the p-doped gallium nitride GaN substrate 160 is etched to allow light L to pass. It is also useful to first of all etch the substrate and then the edge of the semiconductor optical amplifier SOA to create a beveled edge. The blue light LA is amplified in the optical cavity defined by the two beveled edges. After the optogenetic treatment, the retina cells are selectively stimulated by this amplified blue light LA. One of the beveled edges 161 is the input of the signal which is to be amplified. The second 162 is the output of the amplified blue light LA. The beveled edges are inclined at an angle a that is below the limit of the Brewster angle. An anti-reflection coating based on layers of TiO₂ and SiO₂ has been deposited to ensure good optical transmission between the exterior of the device and the guiding layers.

It may be interesting to mix a subretinal prosthesis with an epiretinal prosthesis having the same or a different operating mode. It is also possible to mix the two operating modes, subretinal and epiretinal, in a single retinal prosthesis by the use of metallised holes or vias, to cause the direct and indirect polarisation of the optoelectronic components. It is possible to adapt the structure of multi-quantum wells of the photodiode and the multi-quantum wells of the horizontal cavity of the semiconductor optical amplifier SOA, by the use of butt-joint epitaxy.

Naturally, this invention is not limited to the described embodiments, and is open to many variants accessible to the person skilled in the art in the field without departing from the spirit of the invention. In particular, the composition of the active layer could be modified for any Ill-V semiconductor tuned to the GaN and active in the visible domain, i.e. with a photoluminescence peak in the blue-green-yellow zone.

1. Retinal prosthesis containing a matrix of optoelectronic components including semiconductor optical amplifiers SOAs, the said semiconductor optical amplifiers SOAs containing an active layer of gallium nitride GaN with multiple InGaN/GaAsN (indium-gallium nitride/arsenic-gallium nitride) or InGaN/AlGaN (indium-gallium nitride/aluminium-gallium nitride) quantum wells on a substrate of p-doped gallium nitride GaN and covered with a layer of n-doped gallium nitride GaN.

2. Retinal prosthesis according to claim 1 in which the p-doped gallium nitride GaN substrate forms a column of p-GaN.

3. Retinal prosthesis according to claim 2, in which the column of p-GaN is covered with an insulating layer of biocompatible material chosen from carbon, diamond, titanium dioxide, silica, silicon nitride or gallium nitride.

4. Retinal prosthesis according to claim 2 in which the ratio of the height to the transverse dimension of the p-GaN column is less than 20.

5. Retinal prosthesis according to claim 1 in which the matrix of optoelectronic components contains semiconductor optical amplifiers SOAs with different heights.

6. Retinal prosthesis according to claim 1 in which the matrix of optoelectronic components contains semiconductor optical amplifiers SOAs spaced at a distance E such that E²=π(350/2)²×1/n where n is the number of optoelectronic components in the matrix.

7. Retinal prosthesis according to claim 1 in which the matrix of optoelectronic components contains vertical cavity semiconductor optical amplifiers SOAs or horizontal cavity semiconductor optical amplifiers SOAs.

8. Retinal prosthesis according to claim 7 in which the matrix of optoelectronic components contains at least one vertical cavity semiconductor optical amplifier in which two distributed Bragg reflectors are placed respectively on either side of the active GaN layer with multiple quantum wells in such a way as to define an optical cavity.

9. Retinal prosthesis according to claim 7 in which the matrix of optoelectronic components is a three-dimensional matrix of vertical cavity semiconductor optical amplifiers or horizontal cavity semiconductor optical amplifiers.

10. Retinal prosthesis according to claim 1, in which the matrix of optoelectronic components further contains vertical and horizontal photodiodes.

11. Retinal prosthesis according to claim 10 in which the matrix of optoelectronic components contains vertical or horizontal photodiodes with different heights.

12. Retinal prosthesis according to claim 10, in which the matrix of optoelectronic components contains vertical or horizontal photodiodes spaced at a distance E such that E²=π(350/2)²×1/n where n is the number of optoelectronic components in the matrix.

13. Retinal prosthesis according to claim 1 which is an epiretinal prosthesis.

14. Retinal prosthesis according to claim 1 which is a subretinal prosthesis.

15. Retinal prosthesis according to claim 13 simultaneously containing a subretinal and epiretinal prosthesis.
